(19) United States
(12) Patent Application Publication
(10) Pub. No.: US 2012/0107375 A1
(43) Pub. Date: May 3, 2012

(54) ANTIMICROBIAL MATERIAL

(75) Inventor: Stephen Cotton, Nottinghamshire (GB)

(73) Assignee: BRIGHTWAKE LIMITED, Kirkby-in-Ashfield,Nottinghamshire (GB)

(21) Appl. No.: 13/381,248

(22) PCT Filed: Jul. 1, 2010
(86) PCT No.: PCT/GB10/51088

§ 371 Date: Dec. 28, 2011

(30) Foreign Application Priority Data
Jul. 1, 2009 (GB) 0911402.6

(51) Int. Cl. A61K 36/00; A61P 31/00; A61P 17/02; A61K 9/70

(52) U.S. Cl. 424/400; 424/725

(57) ABSTRACT

An antimicrobial material comprises propolis dispersed within a solid carrier. The antimicrobial material is preferably in the form of a film The carrier may be a polymeric material.

This invention relates to antimicrobial materials and in particular to propolis-containing antimicrobial materials for use in wound care.

An important part of wound care is the prevention of infection. As a result, standard wound treatment methods generally involve the use of antimicrobial agents. However, many such agents have drawbacks, such as cytotoxicity, potentially harming granulation tissue and aggravating wound healing, and poor tolerance by the patient, which may be manifested as fever, nausea or allergic reaction. In addition, in recent years the use of some agents, such as antibiotics, has been discouraged due to the emergence of multi drug resistant microbes, such as MRSA.

The beneficial effects of honey in wound healing are well documented. In particular, honey is a natural, organic material with antiseptic properties, but which is not cytotoxic and is well tolerated by the majority of patients. In general, honey is introduced into the wound space as a liquid and is maintained there by a dressing. However, the natural fluidity of honey, particularly at body temperature, is problematic for the convenient and hygienic handling of antiseptic honey, and makes it difficult to retain sufficient amounts within the wound space.

Propolis, a soluble solid material with substantial antimicrobial properties produced from natural plant resin by bees, is a potential solution to this problem. However, propolis is a brittle solid, making it difficult to work and unsuitable for use as an antiseptic agent in its raw form. There is therefore a need for a suitable means of applying propolis to a wound.

There has now been devised a composition that overcomes or substantially mitigates the above-mentioned and/or other disadvantages associated with the prior art.

According to the invention, there is provided an antimicrobial material comprising propolis dispersed within a solid carrier.

The antimicrobial material is primarily advantageous in that it provides a natural, organic material with antiseptic properties in a form that may be handled conveniently and is particularly suitable for application directly to a wound. The natural, organic nature of the material may mean that it avoids problems such as toxicity and/or environmental damage that may be associated with synthetic and/or inorganic antimicrobial agents such as triclosan or silver.

The antimicrobial material is preferably a conformable solid at room temperature. However, the physical properties of the antimicrobial material depend largely on the choice of carrier. The carrier is preferably capable of forming conformable and flexible sheets that are sufficiently robust to maintain their integrity during normal use and handling. As the antimicrobial material is placed in direct contact with a wound, the carrier is most preferably free from toxic or allergenic substances. The carrier may be formed of a single substance, or may be a composite. The carrier is preferably a polymeric material.

Suitable materials for use as, or in, the carrier include many natural or synthetic polymers. Generally, suitable materials have properties appropriate for use in medical applications, notably absence of toxicity, biocompatibility and, usually, biodegradability. The carrier materials should be such that, in use and once applied to a wound, the carrier dissolves or otherwise breaks down over time, thereby releasing the propolis into the wound environment.

In many embodiments, the carrier comprises a polymer with film-forming properties. A variety of suitable film-forming polymers may be used, provided that they exhibit suitable film-forming properties together with suitability for medical applications, for instance absence of toxicity, biocompatibility and, usually, biodegradability.

Most commonly, the carrier comprises just one polymer, which may be a film-forming polymer. Alternatively, the carrier may comprise a mixture of materials, one or more of which may be a film-forming polymer.

Most preferred carriers are soluble upon exposure to wound fluid. Use of such carriers facilitates the release of propolis into the wound space when the antimicrobial material is in place. The most preferred substance for use as a carrier is polyvinyl alcohol (PVA).

Other synthetic polymers that may be suitable for use in the antimicrobial material are biodegradable polyesters. Specific examples of such polymers are polylactic acid and polyglycolic acid, and copolymers and blends thereof. Other examples include polycaprolactones and polyhydroxyalkanoates, such as polyhydroxybutyrate, polyhydroxyvalerate and polyhydroxyhexanoate.

Further film-forming polymers that may be suitable for use in the antimicrobial material are polysaccharides, and in particular basic polysaccharides.

One such film-forming basic polysaccharide is chitosan, the (at least substantially) N-deacetylated derivative of chitin. Chitin is a naturally abundant mucopolysaccharide and is the supporting material of crustaceans and insects. Chitin is readily obtainable from crustacean shells discarded in the food industry, and the preparation of chitosan from chitin is well known and documented. Derivatives of chitosan may also be used. Chitosan may offer further benefits when used as the carrier material. In particular, chitosan is known to swell when hydrated and so may absorb wound exudate. In addition, chitosan may itself exhibit useful antibacterial and haemostatic properties. Chitosan is also believed to be susceptible to attack by lysozyme, and this may be useful in providing a ready mechanism for biodegradation of the carrier material.

xamples of other synthetic polymers that may be suitable are aminated polymers such as aminated PEGs (including those sold under the trade name JEFFAMINE) and polyallylamines.

Typically, the carrier material is chosen such that the antimicrobial material, in particular the carrier, dissolves or otherwise breaks down over time, so that the propolis is released from it. The material may be such that the antimicrobial material, at least in that part which is in contact with wound fluid, loses its integrity over a timescale of several minutes, for example up to to 10, 30 or 60 minutes, eg 1 to 10 minutes or 10 to 60 minutes, or several hours, for example up to 6, 12 or 24 hours, eg 1 to 6 hours or 6 to 24 hours, or several days, for example up to 1 day or 2 days.

The antimicrobial material may comprise one or more additives, to facilitate production of the material and/or to improve its physical properties. The carrier material and any such additives together generally account for at least 80% w/w of the antimicrobial material, or at least 90% w/w.

One or more additives may be included to improve the physical properties of the antimicrobial material of the invention. For instance, the incorporation of an additive may improve the flexibility and pliability of an antimicrobial sheet according to the invention. One particularly preferred additive is glycerol, which may be present in the antimicrobial material at a level of from about 5% w/w (or 10%) up to about 50% w/w (or 30%), eg about 20% w/w.

Propolis is readily available in the form of a tacky solid, or as a tincture, generally comprising propolis dissolved at relatively high concentration, eg 20 to 50% w/w, in a solvent, eg ethanol . Where solid propolis is used, processing may be required before it is used. For example, raw propolis may be contaminated with solid fragments, such as wood, the presence of which is undesirable in the antimicrobial material. In addition, it may be desirable to sterilise the propolis before it is used to produce the antimicrobial material, to prevent infective agents being incorporated into the antimicrobial material. For this reason, the use of propolis tincture, in which the propolis is generally free of contaminants and infective agents, is preferred.

As propolis is composed mainly of plant resin, its composition is dependent on the plant resins present in the area surrounding the hive at the time. The composition of propolis is therefore variable, depending on geographical location and time of year, as well as the bee species from which it is collected. This variability in the composition of the propolis may potentially cause variation in the properties of the propolis, and therefore in the properties of the propolis-containing antimicrobial material into which it is incorporated. Whilst it is believed that all forms of propolis are potentially beneficial in the antimicrobial material, some forms of propolis may be more efficacious than others.

The antimicrobial material may take any form, including granules, blocks or sheets. However, the antimicrobial material is typically supplied in the form of a flat flexible sheet. This configuration is particularly advantageous, as sheets are easily manipulated, allowing the antimicrobial material to be formed into whatever conformation is required. For example, sheets of antimicrobial material may be cut to the desired shape, stacked, layered, bent, twisted, folded or otherwise deformed to suit a particular application. In addition, antimicrobial material in sheet form may be more conveniently packaged and stored.

Typically, the thickness of the sheets of antimicrobial material is from 20 μm to 5 mm, depending on the intended application of the material. More commonly, the thickness of the sheets of antimicrobial material is from 100 μm to 2 mm, more typically 200 μm to 1 mm.

In presently preferred embodiments of the invention, the antimicrobial material has the form of a thin wafer, typically 100 μm to 500 μm in thickness. Such a wafer is easily handled, and is thin enough to be flexible so that it can conform to the body surface to which it is applied in use.

The content of propolis in the antimicrobial material is typically between 0.5% and 20% w/w, more commonly between about 1% and 10% w/w, eg about 2% to 8% w/w, eg about 4% w/w.

For many applications, sheets of antimicrobial material are preferably provided in sizes that are easy to handle and are appropriate for a single use. Alternatively, or in addition, a sheet of antimicrobial material may be scored or perforated to enable it to be easily cut or torn to a desired size.

For many applications, it is desirable that the antimicrobial material is provided in a sealed bag or sachet to safeguard it from contamination and prevent it drying out and becoming hard and brittle. Such a bag is preferably formed in a moisture-impermeable material such as a metal (eg aluminium) or metallised foil. Alternatively, or in addition, the antimicrobial material in sheet form may be provided with release liners that are removed immediately prior to use.

Because the antimicrobial material is placed in direct contact with a wound, it is preferable that it is sterile to avoid the introduction of infective agents into the wound. Sterilisation may be carried out by any suitable means. However, since the preferred constituents of the antimicrobial material are sensitive to high temperatures, it is preferable that sterilisation is carried out without the use of heat, for example with the use of gamma irradiation or ethylene oxide.

In use, the antimicrobial material is removed from its packaging, cut or otherwise reduced in size if necessary or desired, and placed in direct contact with the wound. The antimicrobial material may then be covered by a suitable dressing to retain it in position.

When the antimicrobial material is in contact with a moist wound surface, the wound fluid causes the propolis to be released from the antimicrobial material. This generates a concentrated propolis solution in the wound space which draws fluid out of the surrounding tissue by osmosis, aiding debridement of the wound and further assisting wound healing. This concentrated propolis solution surrounding the antimicrobial concentrated also prevents it from sticking to the moist wound surface, which would potentially cause pain or damage to granulation tissue.

According to another aspect of the invention, there is provided a method of treating a wound, which method comprises applying to the wound an antimicrobial material comprising propolis dispersed within a solid carrier, or applying to the wound a dressing including such an antimicrobial material.

As well as the use of the antimicrobial material to prevent infection, it may also be used as a therapeutic agent for the treatment of an existing infection. For example, the antimicrobial material may be used as an oral antiseptic to treat conditions such as halitosis, gingivitis and oral ulcers. Antimicrobial material placed in the mouth releases propolis into the surrounding fluid in the same way as in a moist wound space, allowing the propolis to exert its antimicrobial effect.

Thus, another aspect the invention provides a method for the treatment of a microbial infection of the buccal cavity, which comprises the application to a surface of that cavity of an antimicrobial material comprising propolis dispersed within a solid carrier.

In addition, the antimicrobial material may be used in the treatment of cold sores, eg sores caused by the Herpes simplex virus. In this case, the antimicrobial material may be incorporated into a composite article, such as a patch or dressing, which maintains the antimicrobial material in position over the sore. Such a dressing may be of such materials and colour that it is unobtrusive when applied to the skin.

Thus, in a yet further aspect of the invention, there is provided a method for the treatment of a cold sore, which method comprises the application to the cold sore of an antimicrobial material comprising propolis dispersed within a solid carrier, or applying to the cold sore a composite article including such an antimicrobial material.

The antimicrobial material may be incorporated into a composite wound dressing or the like. Therefore, according to a further aspect of the invention, there is provided a wound dressing incorporating an antimicrobial material comprising propolis dispersed within a solid carrier.

In such composite wound dressings, the antimicrobial material may be used in the form of a simple sheet. In other embodiments, however, the antimicrobial material may be used in granular form, or may be used to form bags or sachets that may, for instance, contain a quantity of an absorbent material. Wound dressings incorporating absorbent materials, particularly those known as superabsorbent materials that are capable of absorbing up to several hundred times their weight of water, are well known and often incorporate sachets of permeable material filled with the absorbent material. Use of the antimicrobial material to form such a sachet may enhance the antimicrobial properties of the dressing.

The antimicrobial material may be manufactured in various ways. A currently preferred method of manufacture, which constitutes a further aspect of the invention, comprises the steps of:
a) producing a fluid precursor composition comprising propolis, a carrier material and a liquid medium; and
b) forming that precursor composition into a desired form; and
c) causing or allowing that precursor composition to solidify, to form the antimicrobial material.

The precise composition of the precursor composition may be varied depending on the desired characteristics of the antimicrobial material.

The antimicrobial properties of the antimicrobial material are substantially dependent on the propolis content. Therefore, the propolis content of the precursor composition may be altered depending on the desired antimicrobial properties of the antimicrobial material, or to account for variability in the antimicrobial properties of the propolis itself.

The content of propolis in the precursor composition is chosen according to the desired content of propolis in the antimicrobial material, taking into account that a certain loss of mass may occur during production.

In the currently preferred manufacturing method, PVA is the preferred carrier, and is present in the precursor composition at a level of about 20% w/w to about 75% w/w, eg at a level of about 33% w/w.

In the currently preferred manufacturing method, the precursor composition most preferably contains about 20% w/w to about 80% w/w of liquid medium, eg 66% w/w of liquid medium. The liquid medium is employed in the precursor composition to aid in the mixing of the components and to enable the composition to be cast into the desired form prior to it being caused or allowed to solidify. The liquid medium is preferably non-toxic and non-allergenic, to prevent an adverse reaction when the antimicrobial material is applied to a wound. One particularly preferred liquid medium is glycerol, which may also act as a plasticiser, improving the pliability of the antimicrobial material. Another particularly preferred liquid medium is water. The liquid medium may be a mixture of two different liquids, such as glycerol and water.

The precursor composition may be prepared by dispersing the propolis and carrier material (eg PVA) in the liquid medium. The carrier material is preferably added to the liquid medium in finely divided or powder form and the liquid medium may be heated to aid dispersion. Propolis is preferably added to the liquid medium as a tincture, which may first be combined with an amount of liquid medium to aid mixing. The tincture solvent will generally evaporate as the precursor composition is caused or allowed to solidify.

In one embodiment, the precursor composition may be spread onto a substrate to form a layer of uniform thickness. The substrate may be a film, eg of paper or plastics material, that functions as a release carrier from which the antimicrobial material is peeled or otherwise separated prior to packaging. Alternatively, the release carrier may remain on the finished product, so that it is removed only immediately prior to use. In certain applications, it may not be necessary to remove the release carrier at all.

Once the precursor composition has been cast into the desired form, it is caused or allowed to solidify. This process involves subjecting the precursor composition to different conditions depending on the nature of the carrier material. In particular, this may involve heating the precursor composition for a period of time in order to evaporate some or all of the liquid medium and/or cause the carrier material to set. For instance, the precursor composition may be heated in an oven at a temperature above ambient temperature but generally below about 100° C., eg between 40° and 70° C. for a period of between 5 minutes and 1 hour, eg 15-30 minutes. Heating of the precursor composition generally results in the loss by evaporation of any volatile components, eg the solvent component of any propolis tincture that is used. The liquid medium will generally be at least partially aqueous, and heating will also result in the loss of much of the water that is present. Typically, the antimicrobial material of the invention has a water content of less than 20% w/w, more commonly less than 10%, eg 1 to 20% or 1 to 10% w/w.

The heating process may be carried out batchwise, but is preferably carried out as a continuous process, eg by transporting the precursor composition through an oven on a conveyor.

The method may involve the use of a manufacturing line that is able to continuously form the antimicrobial material. In one such method, a release carrier is fed onto a conveyor, preferably comprising one or more looped belts, which transports it along the manufacturing line. Suction may be applied from beneath the belts to hold the release carrier flat against the surface of the conveyor.

The precursor composition is preferably applied to the release carrier in a uniform layer. This may be carried out by applying the composition from the edge of a suitably formed blade that is positioned close to the release carrier passing beneath it on the conveyor. Following application of the layer of precursor composition, the product is passed through an elongated oven.

Following passage of the product through the oven, the resulting antimicrobial film may then be wound onto a roll for convenient storage or transport. Alternatively, the antimicrobial film may be cut into appropriately sized pieces or formed into granules, which may then be packaged in sealed bags or sachets. The release carrier may be removed prior to packaging, or may remain attached to the product and be removed prior to use.

An embodiment of the invention will now be described in greater detail, by way of illustration only, with reference to the following Example.

### EXAMPLE

### Manufacture and Testing of a Propolis-containing Material

### Manufacture

A carrier composition was produced by dispersing 2.25 kg of PVA in a liquid medium consisting of 0.4 kg of glycerol and 4.5 kg of hot (100° C.) water.

A propolis composition comprising 0.293 kg of propolis tincture (approximately 40% w/w, ie 0.120 kg, propolis in ethanol) and 0.275 kg of glycerol was then added to this carrier composition, giving a precursor composition consisting of 58.3% w/w water, 29.2% w/w PVA, 8.8% w/w glycerol and 3.8% w/w propolis tincture.

The precursor composition was spread onto a release carrier and dried in an oven at 45-65° C. for 20 minutes. Drying resulted in the loss by evaporation of the ethanol component of the propolis tincture and approximately 96% of the water. The resulting final product therefore had a water content of about 6% w/w, and a propolis content of about 3.7% w/w.

The dried product was then wound up onto a roll. Appropriately sized pieces of the product may be cut from the roll and packaged, for instance in sealed foil sachets, and sterilised, or for incorporation into composite products such as wound dressings.

### Microbiological Testing

The product manufactured as described above was cut into pieces with approximate dimensions 10×5 cm².

The test pieces were placed in contact with Tryptone Soya Agar inoculated with a known concentration of one of three organisms (Bacillus subtilis, Staphylococcus aureus and Multiply Resistant S. aureus). Control samples were prepared with the same concentration of organisms.

The number of organisms present (expressed as Colony Forming Units—CFU) were measured as a function of time for the test samples and the controls. The results were as set out in Table 1.

TABLE 1

| | Time/hours | | | | | | | |
|---|---|---|---|---|---|---|---|---|
| | 0 | | 4 | | 24 | | 48 | |
| Organism | Number of organisms (CFU) | | | | | | | |
| used | Product | Control | Product | Control | Product | Control | Product | Control |
| B. subtilis | 800 | 656 | 172 | 952 | 0 | 680 | 0 | 320 |
| S. aureus | 2016 | 1936 | 5 | 2368 | 0 | 1648 | 0 | 1344 |
| MRSA | 1248 | 1456 | 10 | 1456 | 0 | 896 | 0 | 672 |

It can be seen that the propolis-containing product was very effective at reducing levels of bacteria. After 24 hours contact with the product, no viable bacteria were detected.

1. An antimicrobial material comprising propolis dispersed within a solid carrier.

2. An antimicrobial material as claimed in claim 1, which is in the form of a film.

3. An antimicrobial material as claimed in claim 2, wherein the film is a conformable solid at room temperature.

4. An antimicrobial material as claimed in claim 1, wherein the carrier comprises a polymeric material.

5. An antimicrobial material as claimed in claim 1, wherein the carrier comprises a polymer with film-forming properties.

6. An antimicrobial material as claimed in claim 1, wherein the carrier is soluble upon exposure to wound fluid.

7. An antimicrobial material as claimed in claim 6, wherein the carrier comprises polyvinyl alcohol.

8. An antimicrobial material as claimed in claim 1, wherein the propolis content is between 0.5% and 20% w/w.

9. An antimicrobial material as claimed in claim 1, wherein the carrier comprises one or more additives to facilitate production of the material and/or to improve its physical properties.

10. An antimicrobial material as claimed in claim 1, wherein the carrier accounts for at least 80% w/w of the material.

11. An antimicrobial material as claimed in claim 1, wherein the carrier comprises glycerol.

12. An antimicrobial material as claimed in claim 11, wherein the glycerol is present at a level of from about 5% w/w to about 50% w/w.

13. An antimicrobial material as claimed in claim 2, wherein the film has a thickness of between 20 μm and 5 mm.

14. An antimicrobial material as claimed in claim 13, wherein the film is scored or perforated to enable it to be easily cut or torn to a desired size.

15. An antimicrobial material as claimed in claim 1, which is provided in a sealed bag or sachet.

16. An antimicrobial material as claimed in claim 1, which is provided with release liners that are removed prior to use of the material.

17. A method of treating a wound, which method comprises applying to the wound an antimicrobial material as claimed in claim 1.

18. A method of treating an infection, which method comprises applying to the site of infection an antimicrobial material as claimed in claim 1.

19. A method as claimed in claim 18, wherein the infection is a microbial infection of the buccal cavity.

20. A method as claimed in claim 18, wherein the infection is a cold sore.

21. A composite article comprising an antimicrobial material as claimed in claim 1.

22. A method of manufacturing an antimicrobial material as claimed in claim 1, which method comprises:
a) producing a fluid precursor composition comprising propolis, a carrier material and a liquid medium;
b) forming that precursor composition into a desired form; and
c) causing or allowing that precursor composition to solidify, to form the antimicrobial material.

23. A method as claimed in claim 22, wherein the precursor composition contains between 20 and 50% w/w of liquid medium.

24. A method as claimed in claim 22, wherein the liquid medium comprises glycerol.

25. A method as claimed in claim 22, wherein the carrier comprises polyvinyl alcohol.

26. A method as claimed in claim 25, wherein the polyvinyl alcohol content of the precursor composition is from 20% to 75% w/w.

27. A method as claimed in claim 22, wherein the carrier material is added to the liquid medium in finely divided form.

28. A method as claimed in claim 22, wherein the propolis is added to the liquid medium in the form of a tincture.

29. A method as claimed in claim 22, wherein the precursor composition is caused to solidify by heating it in an oven at a temperature between ambient temperature and 100° C. for a period of between 5 minutes and 1 hour.

30. A method as claimed in claim 22, wherein the method is carried out as a continuous process.
